(19) United States
(12) Patent Application Publication
(10) Pub. No.: US 2010/0022887 A1
(43) Pub. Date: Jan. 28, 2010

(54) Method for imaging intracavitary blood flow patterns

(76) Inventors: Joan Carol Main, Scottsdale, AZ (US); A. Jamil Tajik, Fountain Hills, AZ (US); Bijoy Khandheria, Fountain Hills, AZ (US); Farouk Mookadam, Scottsdale, AZ (US); Steven J. Lester, Scottsdale, AZ (US); Ronald J. Buono, Cave Creek, AZ (US)

Correspondence Address: QUARLES & BRADY LLP, 411 E. WISCONSIN AVENUE, SUITE 2040, MILWAUKEE, WI, 53202-4497, US

(21) Appl. No.: 12/460,480

(22) Filed: Jul. 20, 2009

Related U.S. Application Data
(60) Provisional application No. 61/135,486, filed on Jul. 21, 2008.

(51) Int. Cl. A61B 8/06

(52) U.S. Cl. 600/454; 600/458

(57) ABSTRACT

A method for identifying blood flow patterns based on contrast-enhanced ultrasound imaging and, in particular, echocardiography. The method includes indicating a blood flow type in the cavity through which imaged blood is flowing by correlating the identified blood flow pattern with a selected pattern. Further, a report indicative of a condition of the cavity can be generated based on the indicated blood flow type.

### CROSS-REFERENCE TO RELATED APPLICATIONS

This application is based on, claims the benefit of, and incorporates herein by reference U.S. Provisional Application Ser. No. 61/135,486, filed Jul. 21, 2008, and entitled “Method for Imaging Intracavitary Blood Flow Patterns.”

### BACKGROUND OF THE INVENTION

The field of the invention is medical imaging using vibratory energy, such as ultrasound and, in particular, ultrasound imaging of the heart.

The pericardium is a relatively avascular fibrous sac that surrounds the heart and includes two layers, the visceral and parietal pericardia. The visceral pericardium is composed of a single layer of mesothelial cells that adhere to the cardiac epicardium. The parietal pericardium is a fibrous structure typically less than 2 mm thick and composed primarily of collagen with a lesser amount of elastin. The two layers of the pericardium are separated by a potential space that normally contains 15-35 milliliters (mL) of serous fluid, which is mostly distributed over the atrial-ventricular and interventricular grooves. Due to its relative inelasticity, the pericardium limits acute cardiac dilatation and enhances mechanical interactions of the cardiac chambers. The pericardium can dilate in response to long-standing stress, thereby shifting the pericardial pressure-volume relation substantially to the right. This allows a slowly accumulating pericardial effusion to grow significantly without compressing the cardiac chamber and for left ventricular remodeling to occur without pericardial constriction. Pericardial constriction occurs when a scarred, thickened, and frequently calcified pericardium impairs cardiac filling. The most frequent causes are mediastinal radiation, chronic idiopathic pericarditis after cardiac surgery, and tubercutosis pericarditis. Patients with pericardial constriction typically exhibit elevated systemic venous pressures and low cardiac output. Because there is equalization of all cardiac pressure, including both right and left atrial pressure, systemic congestion is much more marked than pulmonary congestion.

Ultrasound, and more particularly Doppler echocardiography, is important in the evaluation of patients with suspected pericardial constriction. There are a number of modes in which ultrasound can be used to produce images of objects. The ultrasound transmitter may be placed on one side of the object and the sound transmitted through the object to the ultrasound receiver placed on the other side (“transmission mode”). With transmission mode methods, an image may be produced in which the brightness of each pixel is a function of the amplitude of the ultrasound that reaches the receiver (“attenuation” mode), or the brightness of each pixel is a function of the time required for the sound to reach the receiver (“time-of-flight” or “speed of sound” mode). In the alternative, the receiver may be positioned on the same side of the object as the transmitter and an image may be produced in which the brightness of each pixel is a function of the amplitude or time-of-flight of the ultrasound reflected from the object back to the receiver (“refraction”, “backscatter” or “echo” mode). The present invention relates primarily to a backscatter method for producing ultrasound images.

There are a number of well known backscatter methods for acquiring ultrasound data. In the so-called “A-mode” scan method, an ultrasound pulse is directed into the object by the transducer and the amplitude of the reflected sound is recorded over a period of time. The amplitude of the echo signal is proportional to the scattering strength of the refractors in the object and the time delay is proportional to the range of the refractors from the transducer. In the so-called “B-mode” scan method, the transducer transmits a series of ultrasonic pulses as it is scanned across the object along a single axis of motion. The resulting echo signals are recorded as with the A-mode scan method and their amplitude is used to modulate the brightness of pixels on a display. The location of the transducer and the time delay of the received echo signals locate the pixels to be illuminated. With the B-mode scan method, enough data are acquired from which a two-dimensional image of the refractors can be reconstructed. Rather than physically moving the transducer over the subject to perform a scan it is more common to employ an array of transducer elements and electronically move an ultrasonic beam over a region in the subject.

The “M-mode” scan method is also known by its full name, “motion mode.” An M-mode scan captures returning echoes signals in only one line of a B-mode image but displays them over a time axis. Movement of structures positioned in that line can then be visualized over time. Often M-mode and B-mode are displayed together on the ultrasound monitor.

In addition, the latest ultrasound systems can now employ 3-D real-time imaging in echocardiograms. Using pulsed or continuous wave Doppler ultrasound, an echocardiogram can also produce accurate assessment of the velocity of blood or tissue at any chosen point. Doppler systems employ an ultrasonic beam to measure the velocity of moving reflectors, such as flowing blood cells or tissue. Blood velocity or tissue velocity is detected by measuring the Doppler shifts in frequency imparted to ultrasound by reflection from moving blood cells or tissue. Accuracy in detecting the Doppler shift at a particular point depends on defining a small sample volume at the required location and then processing the echoes to extract the Doppler shifted frequencies.

Echocardiography can provide information on the pericardial condition, but it can be difficult to make a differential diagnosis in the presence of complicated disease processes. Doppler echocardiography frequently depicts restricted filling of both ventricles with a rapid deceleration of the early diastolic mitral inflow velocity (E-wave) and small or absent A-wave. Other findings in the constrictive pericarditis include preserved diastolic Mitral annular velocity, rapid diastolic flow propagation to the apex, and diastolic Mitral regurgitation. Echocardiography is also useful in differentiating pericardial constriction from right heart failure due to tricuspid valve disease or associated pulmonary hypertension. It is more difficult to differentiate between pericardial constriction and restrictive cardiomyopathy. Rapid propagation of early diastolic flow to the apex is preserved in constriction and reduced in restriction. A slope greater than or equal to 100 cm/s of the first aliasing contour in the color M-mode has been used to distinguish the two conditions.

A significant problem in ultrasonic imaging is that many of the body's internal structures have similar acoustic impedance properties, leading to inadequate contrast between structures of interest in ultrasound images. In particular, the muscles of the heart are perfused with blood, which further confounds different structures and makes it difficult to distinguish between blood vessels, heart chambers, and the heart muscle. Ultrasonic contrast agents can be employed to address this problem. These contrast agents include small bubbles of gas, such as air, formed by agitating a liquid or bubbling gas through a liquid, such as a saline, or a solution containing a bubble forming compound, such as albumin. When insonified, the bubbles resonate at their resonant frequency and emit energy at both the fundamental and second harmonics of their resonant frequency, thereby returning an enhanced signal at or around these frequencies and providing an contrast enhanced image of the tissue of interest. It can still be difficult to distinguish or differentiate between various pathologies, even when using a contrast agent. This is particularly problematic when attempting to discern between pericardial constriction and right heart failure due to tricuspid valve disease or associated pulmonary hypertension.

It would thus be desirable to have a system and method to aid in the review, analysis, and diagnosis of heart conditions using non-invasive tools, such as ultrasound imaging.

### SUMMARY OF THE INVENTION

The present invention overcomes the aforementioned drawback by providing a non-invasive method for identifying blood flow patterns and analyzing a condition associated with of a cavity being imaged based on the blood flow pattern. This method comprises the steps of acquiring ultrasound data from a region-of-interest (ROI) in a subject having been administered an ultrasound contrast agent, wherein the ROI includes a cavity through which blood containing the contrast agent flows, automatically identifying a blood flow pattern in the cavity from the ultrasound data, automatically correlating the identified blood flow pattern with a selected flow pattern to provide an indication of blood flow type, and generating an image of the ROI from the acquired ultrasound data in which at least one of the identified blood flow pattern and indicated blood flow type is visually indicated.

The invention is not limited to these aspects, and various other features of the present invention will be made apparent from the following detailed description and the drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram of an ultrasonic imaging system that employs the present invention;

FIG. 2 a block diagram of a receiver that forms part of the system of FIG. 1;

FIG. 3 is a flow chart of a method for identifying and displaying flow patters indicative of different disease states in relationship to the 3D flow patterns of the normal subject using the system of FIG. 1; and

FIG. 4 is a contrast-enhanced color coded M-mode image including markers depicting flow characteristics in accordance with the present invention.

### DETAILED DESCRIPTION OF THE INVENTION

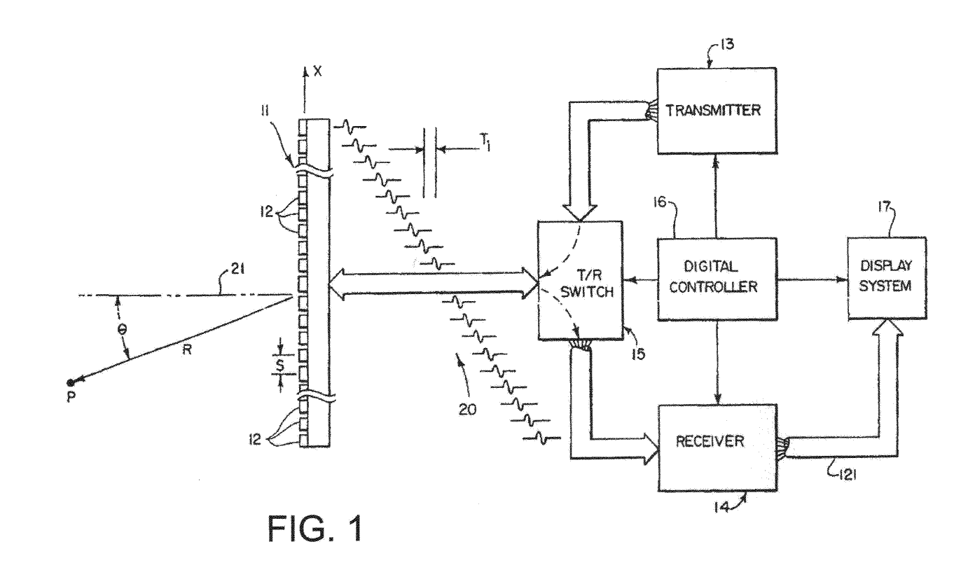

Referring particularly to FIG. 1, an ultrasonic imaging system includes a transducer array 11 comprised of a plurality of separately driven elements 12 that each produce a burst of ultrasonic energy when energized by a pulse produced by a transmitter 13. The ultrasonic energy reflected back to the transducer array 11 from the subject under study is converted to an electrical signal by each transducer element 12 and applied separately to a receiver 14 through a set of switches 15. The transmitter 13, receiver 14 and the switches 15 are operated under the control of a digital controller 16 responsive to the commands input by the human operator. A complete scan is performed by acquiring a series of echoes in which the switches 15 are set to their transmit position, the transmitter 13 is gated on momentarily to energize each transducer element 12, the switches 15 are then set to their receive position, and the subsequent echo signals produced by each transducer element 12 are applied to the receiver 14. The separate echo signals from each transducer element 12 are combined in the receiver 14 to produce a single echo signal that is employed to produce a line in an image on a display system 17.

The transmitter 13 drives the transducer array 11 such that the ultrasonic energy produced is directed, or steered, in a beam. A B-scan can therefore be performed by moving this beam through a set of angles from point-to-point rather than physically moving the transducer array 11. To accomplish this, the transmitter 13 imparts a time delay (Tᵢ) to the respective pulses 20 that are applied to successive transducer elements 12. If the time delay is zero (Tᵢ=0), all the transducer elements 12 are energized simultaneously and the resulting ultrasonic beam is directed along an axis 21 normal to the transducer face and originating from the center of the transducer array 11. As the time delay (Tᵢ) is increased as illustrated in FIG. 1, the ultrasonic beam is directed downward from the central axis 21 by an angle θ. A sector scan is performed by progressively changing the time delays Tᵢ in successive excitations. The angle θ is thus changed in increments to steer the transmitted beam in a succession of directions.

Referring still to FIG. 1, the echo signals produced by each burst of ultrasonic energy emanate from reflecting objects located at successive positions (R) along the ultrasonic beam. These are sensed separately by each segment 12 of the transducer array 11 and a sample of the magnitude of the echo signal at a particular point in time represents the amount of reflection occurring at a specific range (R). Due to the differences in the propagation paths between a focal point P and each transducer element 12, however, these echo signals will not occur simultaneously and their amplitudes will not be equal. The function of the receiver 14 is to amplify and demodulate these separate echo signals, impart the proper time delay to each and sum them together to provide a single echo signal that accurately indicates the total ultrasonic energy reflected from each focal point P located at range R along the ultrasonic beam oriented at the angle θ.

To simultaneously sum the electrical signals produced by the echoes from each transducer element 12, time delays are introduced into each separate transducer element channel of the receiver 14. In the case of the linear array 11, the delay introduced in each channel may be divided into two components, one component is referred to as a beam steering time delay, and the other component is referred to as a beam focusing time delay. The beam steering and beam focusing time delays for reception are precisely the same delays (Tᵢ) as the transmission delays described above. However, the focusing time delay component introduced into each receiver channel is continuously changing during reception of the echo to provide dynamic focusing of the received beam at the range R from which the echo signal emanates.

Under the direction of the digital controller 16, the receiver 14 provides delays during the scan such that the steering of the receiver 14 tracks with the direction of the beam steered by the transmitter 13, and it samples the echo signals at a succession of ranges and provides the proper delays to dynamically focus at points P along the beam. Thus, each emission of an ultrasonic pulse results in the acquisition of a series of data points that represent the amount of reflected sound from a corresponding series of points P located along the ultrasonic beam.

The display system 17 receives the series of data points produced by the receiver 14 and converts the data to a form producing the desired image. For example, if an A-scan is desired, the magnitude of the series of data points is merely graphed as a function of time. If a B-scan is desired, each data point in the series is used to control the brightness of a pixel in the image, and a scan comprised of a series of measurements at successive steering angles (θ) is performed to provide the data necessary for display.

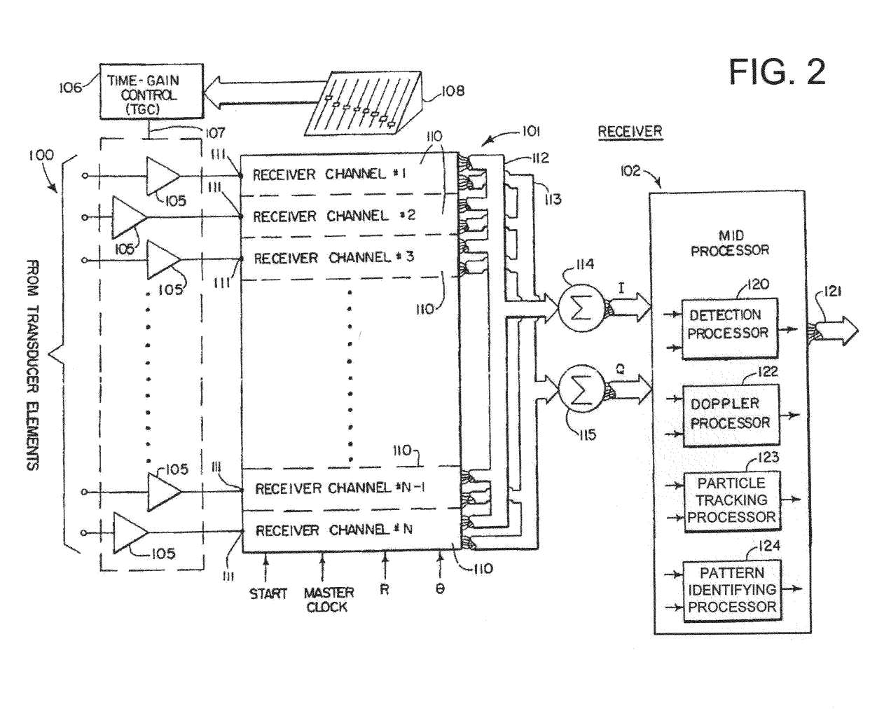

Referring particularly to FIG. 2, the receiver 14 is comprised of three sections: a time-gain control section 100, a beam forming section 101, and a mid processor 102. The time-gain control section 100 includes an amplifier 105 for each of, for example, N=128 receiver channels and a time-gain control circuit 106. It is noted that 128 receiver channels is selected for exemplary purposes and that other numbers of channels are contemplated. The input of each amplifier 105 is connected to a respective one of the transducer elements 12 to receive and amplify the echo signal that it receives. The amount of amplification provided by the amplifiers 105 is controlled through a control line 107 that is driven by the time-gain control circuit 106. As is well known in the art, as the range of the echo signal increases, its amplitude is diminished. As a result, unless the echo signal emanating from more distant reflectors is amplified more than the echo signal from nearby reflectors, the brightness of the image diminishes rapidly as a function of range (R). This amplification is controlled by the operator who manually sets eight (typically) time gain compensation (TGC) linear potentiometers 108 to values that provide a relatively uniform brightness over the entire range of the sector scan. The time interval over which the echo signal is acquired determines the range from which it emanates, and this time interval is divided into eight segments by the TGC control circuit 106. The settings of the eight potentiometers are employed to set the gain of the amplifiers 105 during each of the eight respective time intervals so that the echo signal is amplified in ever increasing amounts over the acquisition time interval.

The beam forming section 101 of the receiver 14 includes separate receiver channels 110. Each receiver channel 110 receives the analog echo signal from one of the TGC amplifiers 105 at an input 111, and it produces a stream of digitized output values on an “I” bus 112 and a “Q” bus 113. Each of these I and Q values represents a sample of the echo signal envelope at a specific range (R). These samples have been delayed in the manner described above such that when they are summed at summing points 114 and 115 with the I and Q samples from each of the other receiver channels 110, they indicate the magnitude and phase of the echo signal reflected from a point P located at range R on the steered beam (θ).

Referring still to FIG. 2, the mid processor section 102 receives the beam samples from the summing points 114 and 115. The “I” and “Q” values of each beam sample is a 16-bit digital number that represents the in-phase and quadrature components of the magnitude of the reflected sound from a point (R, θ). The mid processor 102 can perform a variety of calculations on these beam samples, where choice is determined by the type of image to be reconstructed. For example, if a conventional magnitude image is to be produced, a detection process indicated at 120 is implemented in which a digital magnitude M is calculated from each beam sample and output at 121.

M=\sqrt{I²+Q²}

The mid processor may also include a Doppler processor 112. Such Doppler processors often employ the phase information (φ) contained in each beam sample to determine the velocity of reflecting objects along the direction of the beam (i.e. radial direction from the center of the transducer 11, where φ=tan⁻¹ (I/Q).

The mid processor 102 may also include a particle tracking processor 123. The particle tracking processor 123 is similar to what is referred to in the art as particle image velocimetry (“PIV”). In this application, the particle tracking processor detects the microbubble contrast agent particles by their characteristic acoustic signature in successive image frames and calculates therefrom their displacement vector and velocity. Other quantitative blood flow parameters such as acceleration, vorticity, turbulence, circulation, and laminarity can also be calculated for each image frame. Any of these flow parameters may be output to the display where they can be shown in color superimposed over an anatomic, magnitude image of the structure being imaged.

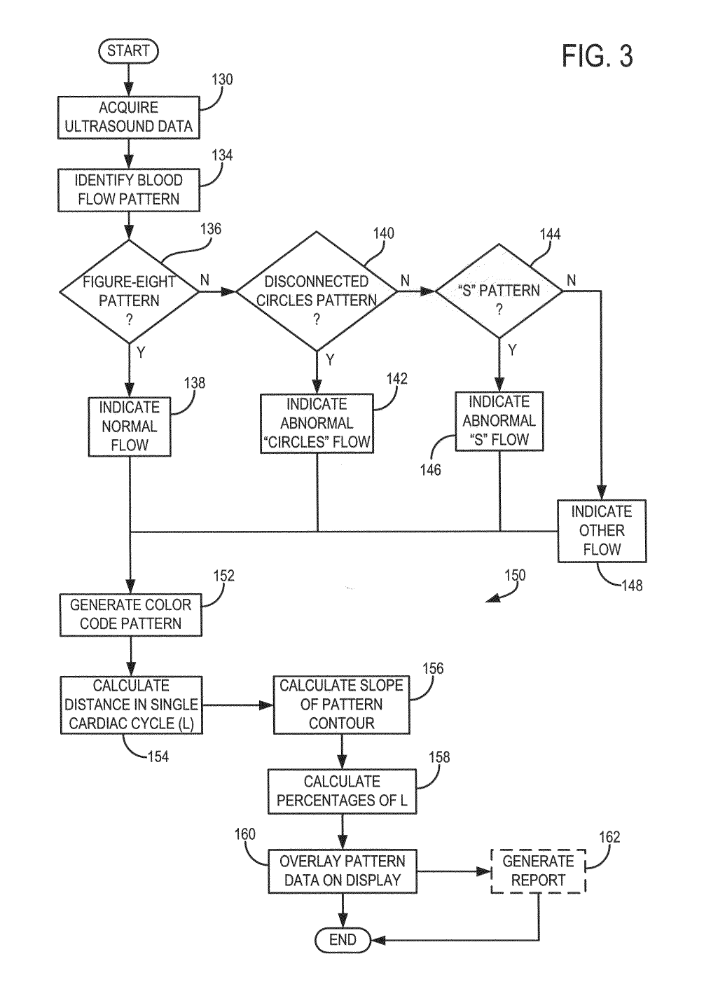

Also, as will be described, the present invention includes a pattern identifying processor 124. The pattern identifying processor 124 may be a “virtual processor” and need not be a physical processor separate from other “processors” in the mid processor 102. As will be described in further detail with respect to FIG. 3, the pattern identifying processor 124 is designed to identify flow patterns indicative of wall mechanics of an imaged heart. The flow patters may be superimposed over traditional images or other traditional information displays to illustrate, in real time, the heart wall mechanics of the imaged individual.

Referring now to FIG. 3, a method in accordance with the present invention begins at process block 130 with the acquisition of ultrasound data from a subject having been administered a contrast agent and the production of an ultrasound image, such as an echocardiogram. In particular, contrast agents having particles such as microbubbles that act as blood tracers and can be visualized using high-temporal resolution ultrasound may be beneficial for imaging cardiac cavities and blood vessels. For example, the perfluoropropane gas-filled, lipid-stabilized microbubble contrast agent “Definity,” such as available from Bristol-Myers Squibb Medical Imaging Inc., North Billerica, Mass., may be used. Definity is a registered trademark of Lantheus Medical Imaging, Inc. Corp. of Delaware.

Following the production of the echocardiogram, an M-mode contour of blood flow patterns in an M-mode display is automatically detected at process block 134 using, for example, the pattern identifying processor 124 of FIG. 2. Because cardiac wall mechanics change with pressure, the mechanical properties of the cardiac wall can be indirectly assessed by analyzing blood flow patterns. Accordingly, constrictive and restrictive processes, as well as changes in flow direction can be recorded in an M-mode image. Processing algorithms can be employed to identify complex patterns within the M-mode image and correlate the patterns with specific pathologies. As will be described, the identified patterns are correlated to specific flow patters that are indicative of known pathologies.

At decision block 136, the identified pattern is compared to a selected “figure-eight” pattern. If the identified pattern correlates well to the selected “figure-eight” pattern, such as having a correlation metric that is above a selected threshold, then a normal blood flow pattern is indicated at process block 138 and, as will be described later, a color code pattern is generated at process block 152. For example, during the correlation comparison, the pattern identifying process 124 of FIG. 2 varies a predetermined figure-eight pattern, such as in size and other dimensional relationships to attempt to correlate the identified patter with the predetermined figure-eight pattern. The degree of variation from the predetermined figure-eight pattern can be translated into a “score” or correlation metric that is then compared to a threshold value. The threshold value may be specific to a given pattern or may be standardized across all patterns.

If the correlation is poor or below a selected threshold, then the identified pattern is compared to a selected “disconnected circles” pattern at decision blood 140. Should the identified patterns correlate well to the “disconnected circles” pattern, then an abnormal “circles” flow is indicated at process block 142 and a color code pattern is generated at process block 152. If poor correlation between the identified pattern and the selected “disconnected circles” pattern is found, then the identified pattern is compared to a selected “S” pattern at decision block 148. Abnormal “S” flow is indicated at process block 146 if the identified pattern and the “S” pattern correlate well, while other abnormal flow is indicated at process block 148 if the identified pattern and the “S” pattern correlate poorly. In either case, a color code pattern is generated at process block 150 following flow state indication.

As indicated generally at 150, color code pattern generation is part of a series of data processing and analysis steps. Like the preceding data acquisition, pattern identification, and flow indication steps, these data processing and analysis steps can be performed in real time or during post processing. The color code pattern can be used to visually accentuate the identified blood flow patterns in an M-mode waterfall image. At process block 154, the distance or length (L) of a single cardiac cycle in the image is calculated and, using this information, the slope of the identified pattern contour is calculated at process block 156. Subsequently, image regions corresponding to 60 percent and 40 percent of L are identified at process block 158. As indicated process block 160, an image is generated in which the generated color code pattern is superimposed with the M-mode waterfall image. This provides a visually intuitive display image for an operator, since image regions corresponding to an identified blood flow pattern have a distinct color coding and can be readily identified via visual inspection. The calculated parameters, such as the slope and cardiac cycle length, can also be included in the image for quick assessment by the operator. It is also contemplated that data can be communicated in a parametric display of flow vortices within the ventricle, which can be enhanced using contrast agents for left ventricular opacification and endocardia border detection. Since certain blood flow types relate to certain cavity conditions, as described above, a report indicative of the condition of the cavity can optionally be generated at process block 162. For example, when imaging the heart, a report may be generated in which identified abnormal blood flow types are related to known cardiac conditions.

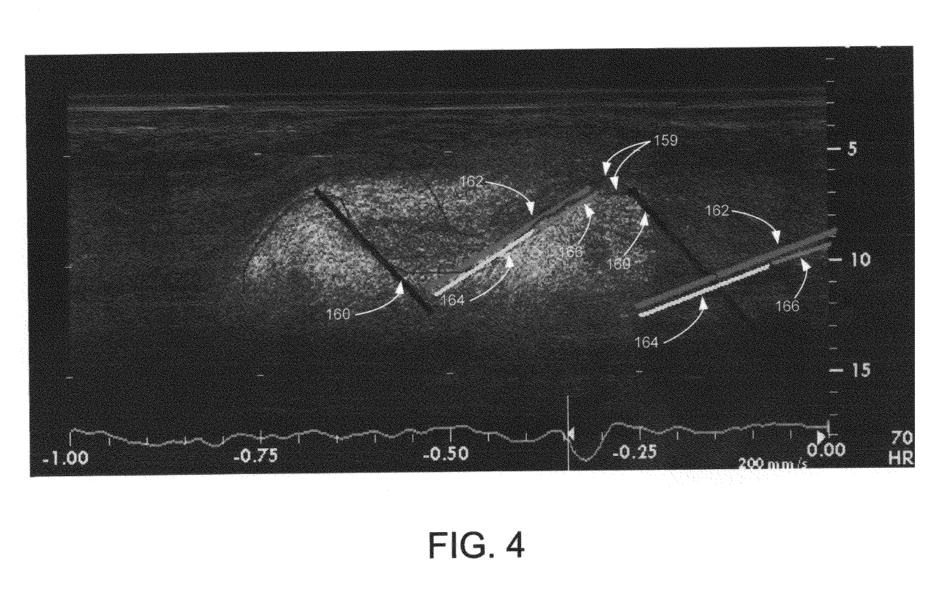

FIG. 4 provides an exemplary contrast-enhanced M-mode waterfall image showing a color-coded blood flow pattern 159 generated in accordance with the present invention. The image also includes an acceleration slope marker 160, a length (L) marker 162, and the markers 164 and 166 which correspond to 60 and 40 percent of L, respectively. Similar images in which the identified blood flow pattern or calculated parameters are communicated differently can also be generated in accordance with the present invention.

M-mode imaging of a contrast agent in the ventricles is beneficial for displaying flow patterns in specific patient populations. The above-described M-mode image processing and display can be applied to any 2D or 3D ultrasound application using contrast agent and is angle independent. Furthermore, it can readily be utilized with 3D waterfall displays. By correlating patterns with Doppler measurements and color flow M-mode propagation, quantitative analysis of pathological states is enabled. This approach can potentially allow early disease management and improvements clinical workflow, since its non-invasiveness allow an operator to examine a subject regularly. It should be noted that the order and particulars of above-described method can be altered and still produce images and indications of a condition in a cavity in accordance with the present invention. For example, the steps of correlating and subsequently indicating the various flow states could be performed in a different order or in parallel.

The present invention has been described in terms of the various embodiments, and it should be appreciated that many equivalents, alternatives, variations, and modifications, aside from those expressly stated, are possible and within the scope of the invention. Therefore, the invention should not be limited to a particular described embodiment.

1. A method for imaging blood flow patterns in a subject, the method comprising the steps of:
a) acquiring ultrasound data from a region-of-interest (ROI) in a subject having been administered an ultrasound contrast agent, wherein the ROI includes a cavity through which blood containing the contrast agent flows;
b) automatically identifying a blood flow pattern in the cavity from the ultrasound data;
d) automatically correlating the identified blood flow pattern with a selected flow pattern to provide an indication of blood flow type; and
e) generating an image of the ROI from the acquired ultrasound data in which at least one of the identified blood flow pattern and indicated blood flow type is visually indicated.

2. The method as recited in claim 1 further comprising f) generating a report indicative of a condition of the cavity based in the indicated blood flow type.

3. The method as recited in claim 1 wherein step d) further includes calculating parameters related to the identified blood flow pattern and step e) further includes displaying the calculated parameters on the image of the ROI.

4. The method as recited in claim 3 wherein the calculated parameters include at least one of a length of a single cardiac cycle, a slope of the identified blood flow pattern, and image locations corresponding to different percentages of the length of the cardiac cycle.

5. The method as recited in claim 4 wherein the different percentages of the length of the cardiac cycle are substantially 60 percent and 40 percent.

6. The method as recited in claim 3 wherein the ROI includes the subject's heart and an abnormal condition of the cavity corresponds to a cardiac abnormality.

7. The method as recited in claim 1 wherein the selected flow pattern is at least one of a figure-eight pattern, disconnected-circles pattern, and S pattern.

8. The method as recited in claim 7 wherein an abnormal blood flow type is indicated in step d) by correlation above a selected threshold of the identified blood flow pattern and at least one of the S pattern and the disconnected-circles pattern.

9. The method as recited in claim 7 wherein a normal blood flow type is indicated in step d) by correlation above a selected threshold of the identified blood flow pattern and the figure-eight pattern.

10. The method as recited in claim 7 wherein an abnormal blood flow type is indicated in step d) by correlation below a selected threshold of the identified blood flow pattern and the disconnect circles pattern, figure-eight pattern, and S pattern.

11. The method as recited in claim 1 wherein steps b)-e) occur substantially in real time.

12. The method of claim 11 wherein step e) includes color-coding the identified blood flow pattern in the image of the ROI.

13. The method as recited in claim 11 wherein the image of the ROI includes a M-mode waterfall image reconstructed from the acquired ultrasound data superimposed with the color-coded blood flow pattern.

14. The method as recited in claim 1 wherein the contrast agent is a perfluoropropane gas-filled, lipid-stabilized microbubble contrast agent.
